(19) United States
(12) Patent Application Publication
(10) Pub. No.: US 2015/0018724 A1
(43) Pub. Date: Jan. 15, 2015

(54) Balance Augmentation Sensors

(71) Applicants: Ying Hsu, San Clemente, CA (US); Ellwood Payson, Fullerton, CA (US); Itzhak Sapir, Irvine, CA (US); Virgilio Villacorta, Corona, CA (US)

(72) Inventors: Ying Hsu, San Clemente, CA (US); Ellwood Payson, Fullerton, CA (US); Itzhak Sapir, Irvine, CA (US); Virgilio Villacorta, Corona, CA (US)

(21) Appl. No.: 14/331,686

(22) Filed: Jul. 15, 2014

Related U.S. Application Data
(60) Provisional application No. 61/846,237, filed on Jul. 15, 2013.

(51) Int. Cl. A61B 5/11

(52) U.S. Cl. CPC A61B 5/1116; A61B 5/1121; A61B 5/112
USPC 600/595

(57) ABSTRACT

Current rehabilitative treatment of balance disorder requires long and intensive processes. The development of a wearable device for use in clinic as well as at home and during daily routine activities would enable continuous treatment outside of specialized training settings and accelerated relieve of balance disorder. The patent describes a wearable device that utilizes inertial sensors and sensor fusion processing to measure body posture and provide real-time feedback to alert the wearer to remain in the region of stability, and that utilizes wireless communication to support therapy and training providers.

### CROSS-REFERENCE TO RELATED APPLICATIONS

This application claims the benefit of U. S. Provisional Patent Application No. 61/846,237, filed on Jul. 15, 2013 entitled “Balance Augmentation Sensor” pursuant to 35 USC 119, which application is incorporated fully herein by reference.

### STATEMENT REGARDING FEDERALLY SPONSORED RESEARCH AND DEVELOPMENT

N/A

### BACKGROUND OF THE INVENTION

1. Field of the Invention

The invention relates generally to the field of electronic sensor systems. More specifically, the invention relates to a balance augmentation sensor method and device for human balance awareness and rehabilitation.

2. Background of the Invention

Development of wearable devices for balance disorder rehabilitation has been well-researched with some devices available commercially. Although several sensory augmentation techniques (or balance prostheses) have been reported, including vibro-tactile on the body and legs, auditory feedback, tongue-placed tactile, and galvanic stimulation (sending electric stimulation to a nerve in the ear), the tactile feedback approach of the instant invention represents an ideal sensory augmentation technique. This is because tactile feedback can be easily placed on and removed from a user, thus lending itself to a solution of using a small and unobtrusive wearable device.

By making the sensory device small and lightweight, the disclosed invention has the advantage over prior art devices of having the ability to be placed anywhere on the body (trunk, limbs and head), providing sensing as well as tactile feedback, and having wireless communication using a processing computer or in cooperation with other sensors.

### BRIEF SUMMARY OF THE INVENTION

The patent describes a wearable device that utilizes inertial sensors and sensor fusion processing to measure body posture and provide real-time feedback to alert the wearer to remain in the region of stability, and that utilizes wireless communication to support therapy and training providers.

These and various additional aspects, embodiments and advantages of the present invention will become immediately apparent to those of ordinary skill in the art upon review of the Detailed Description and any claims to follow.

While the claimed apparatus and method herein has or will be described for the sake of grammatical fluidity with functional explanations, it is to be understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112, are to be accorded full statutory equivalents under 35 USC 112.

### BRIEF DESCRIPTION OF THE SEVERAL VIEWS OF THE DRAWINGS

FIG. 1 depicts the Balance Augmentation Sensors (BATS).

FIG. 2 illustrates how BATS may be worn by users for rehabilitation treatment.

FIG. 3 depicts a flow diagram of BATS operations.

FIG. 4 depicts preferred block diagram of the BATS system.

FIG. 5 shows two suitable chip-scale and module IMUs that are commercially available for use in the invention.

FIG. 6 illustrates the expected angle output over time using a precise module gyroscope versus a chip-scale gyroscope.

FIG. 7 illustrates how bias instability error is cancelled in the device and method of the invention.

FIG. 8 shows how a body's angular velocities exhibit both positive and negative direction.

FIG. 9 illustrates the use of both an accelerometer and gyroscope to measure the body tilt over short time scales (gyroscope) and over longer time scales (accelerometer).

The invention and its various embodiments can now be better understood by turning to the following detailed description of the preferred embodiments which are presented as illustrated examples of the invention defined in the claims.

It is expressly understood that the invention as defined by the claims may be broader than the illustrated embodiments described below.

### DETAILED DESCRIPTION OF THE INVENTION

There is a need for a wearable medical device to support rehabilitation treatment of human balance disorders. One application for such a device is the rehabilitation of injured warfighters suffering from injury-induced balance disorders to assist in their return to duty and to improve their quality of life while living with the impairment.

A short-duration course of balance rehabilitation treatment is desirable which duration is reduced by combining a rehabilitation program with the use of a wearable device to accelerate and permanently relieve the balance disorder. By providing uninterrupted patient feedback as part of the balance disorder treatment, a shorter recovery time and relief of the disorder are achievable.

The science of balance control and rehabilitation supplemented with sensory augmentation techniques is well-researched and several wearable devices are commercially available or are in different development stages. However, any requirement for using the device outside of clinical settings presents significant challenges.

First, prior art balance augmentation devices are cumbersome to use since they are designed mainly for medical diagnostic and training purposes.

Second, prior art sensor devices simply cannot maintain high accuracy over long measurement durations or in outdoor environments with varying temperatures and humidity.

Third, current balance control technology does not employ intelligent algorithms that timely adapt to users' activity and adjust sensory feedback for optimum rehabilitation.

Lastly, prior art devices do not wirelessly communicate with smart phones or internet technology to provide instant feedback to a user at anytime and anywhere.

A balance augmentation device that addresses the above deficiencies in the prior art would desirably entail providing a small, easy to use, and unobtrusive wearable device that not only provides effective balance control, but also motivates behavioral changes to enhance accelerated relief of balance disorders.

To address the above deficiencies, Applicant discloses a “Balance Augmentation and Tracking Sensor” (“BATS” herein).

The BATS of the invention is provided as a miniature sensor device designed to measure human body trunk tilt angle, and to immediately alert a user when the user's trunk tilt exceeds a predefined limit of stability.

The device of the invention may be comprised of a chip-scale inertial measurement unit (IMU) and a plurality of, preferably three, magnetometers, a vibration motor, a wireless communication chip, a processor and a rechargeable battery. The BATS is configured to be clipped onto a belt on the lower back of a user and to measure body tilt angles and velocities.

Key innovations of the invention include at least the following:

1) BATS incorporates low-cost chip-scale IMU and magnetic sensors combined with sensor fusion algorithms to sense accurate body tilt over long measurement times and varying environments,

2) BATS integrates both sensing and tactile functions into a single device and may be configured to communicate wirelessly with other BATS units, smart phone or computers. These innovations and the invention's small size permits BATS to be used in clinical settings as well as providing an easy and unobtrusive device for use at home and during daily routines.

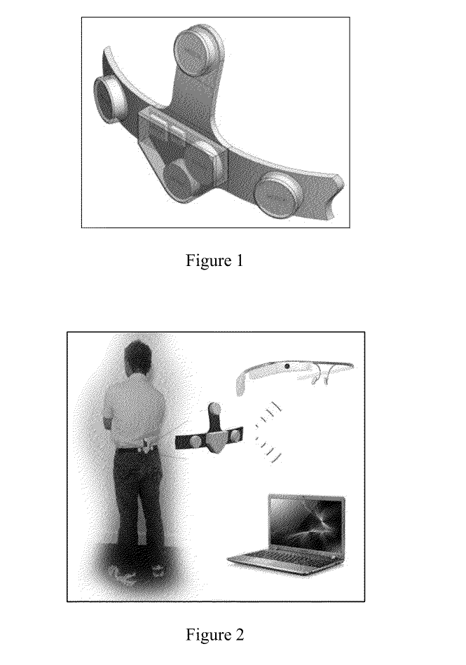

A preferred embodiment of the BATS of the invention is depicted in FIG. 1.

In use, BATS monitors the user's body tilt angle. When the tilt exceeds a pre-set value, the processor of the device activates the unit's vibration motor to cue the user to pull away from the region of instability. To alert the user, the BATS may be configured to vibrate at different frequencies to indicate moving in the positive (e.g., forward) or negative (e.g., backward) directions.

The small and lightweight BATS may be clipped onto the user's belt, inner garment, or placed on the body trunk or limbs using an elastic band. For users that require monitoring and control in multiple directions, several BATS can be used and networked together.

The BATS unobtrusive format ensures the device is convenient and easy to use, while providing clinically beneficial information to assist in balance rehabilitation treatment.

Making the device “easy to use” means that setting up the device involves only clipping the device onto the belt or garment and the built-in sensors are configured to automatically initiate all measurements and communications.

To make BATS clinically useful for balance rehabilitation, the device uses commercially available, chip-scale, and low-cost sensors to accurately measure body tilt, and to do so over long durations and varying temperatures.

FIG. 2 illustrates how BATS may be worn by users for rehabilitation treatment.

Overcoming the limitations of prior art, low-cost but low-stability chip-scale sensors is a technical challenge. The BATS device and method of the invention provides several unique technological advantages, including:

1. Small Sensor Package: A preferred embodiment of the BATS permits a relatively small package volume of about 2.5 cm3 (0.16 in3), with an area that is just slightly larger than a U.S. quarter including the IMU (three accelerometers, three gyroscopes), three magnetic sensors, a wireless communication chip, vibration motor, microprocessor and battery.

2. Lower Power: A power consumption estimate of the preferred embodiment of the BATS shows approximately 63 mW is required for continuous operation. With a small coin cell (10 mm diameter), device operation can last approximately 1.4 hours, while a larger coin cell (LR2450, 25 mm diameter) will last approximately 6.7 hours.

3. Accurate Body Tilt Measurement: The BATS of the invention uses a chip-scale IMU and is capable of measuring body tilt about three axes. For balance control, however, the BATS only needs to measure body tilt about two-axis; i.e., in the anterior-posterior (A/P) and medio-lateral (M/L) directions.

A technical issue in using a prior art chip-scale IMU is overcoming sensor drift that can easily exceed several degrees after only a few seconds. To overcome this bias instability limitation, the BATS herein may incorporate several error compensation techniques, including the use of tri-axial accelerometers to independently determine body tilt in conjunction with a sensor fusion algorithm, active drift cancellation using the body's reciprocal motion, and adopting the use of two oppositely-oriented IMUs.

4. Integrated Sensor and Vibration-Tactile Feedback: The BATS provides sensor augmentation to the user through a vibration motor that serves as tactile (also known and referred to herein as a “tactor”) feedback to alert the user to move his or her body so as to remain in the region of stability. Integrating both the sensor and tactor in a small package herein desirably eliminates cumbersome wiring connecting the various components and greatly simplifies treatment setup. The integrated device may be placed anywhere on the body (with, for instance, an elastic band) and enables location specific measurement and sensory feedback.

The BATS device and method of the instant application enables a new type of balance rehabilitation aid with the at least following benefits:

1. Easy to use/faster recovery: The small size of the BATS makes it easy for the user to put on the device without cumbersome “setup time.” The wireless capabilities of the BATS permits seamless transfer of data to a central processor (smart phone, tablet or computer), and instantly displays useful and easy to understand graphics that allow the user to view his or her progress. The ease of use promotes greater confidence in the device and motivates the user to adhere to the rehabilitation treatment for faster recovery.

2. Continuous rehabilitation: BATS may be used in both clinical settings as well as in the home and during outdoor activities. By employing the same device in different settings (medically supervised and un-supervised), users become familiar with the device and increase their confidence in using BATS in their rehabilitation program. In time, with improvements in their balance and additional reward systems, BATS enables a “continuous” rehabilitation treatment.

A preferred embodiment for the BATS and system design are discussed below.

A number of studies have shown the effectiveness of postural control by measuring body trunk tilt angle and by providing vibro-tactile feedback that guides the patient to remain in the region of balance stability.

A preferred embodiment of the BATS system is depicted in FIG. 2.

The BATS is shown clipped onto the belt of a user, near the lower back. For more accurate measurement of the body's center of mass, the placement may be on the trunk midline (behind navel) at approximately the L4/L5 vertebrate level. Instead of clipping onto the belt, BATS can also be clipped onto an inner garment or to an elastic belt worn over the body trunk.

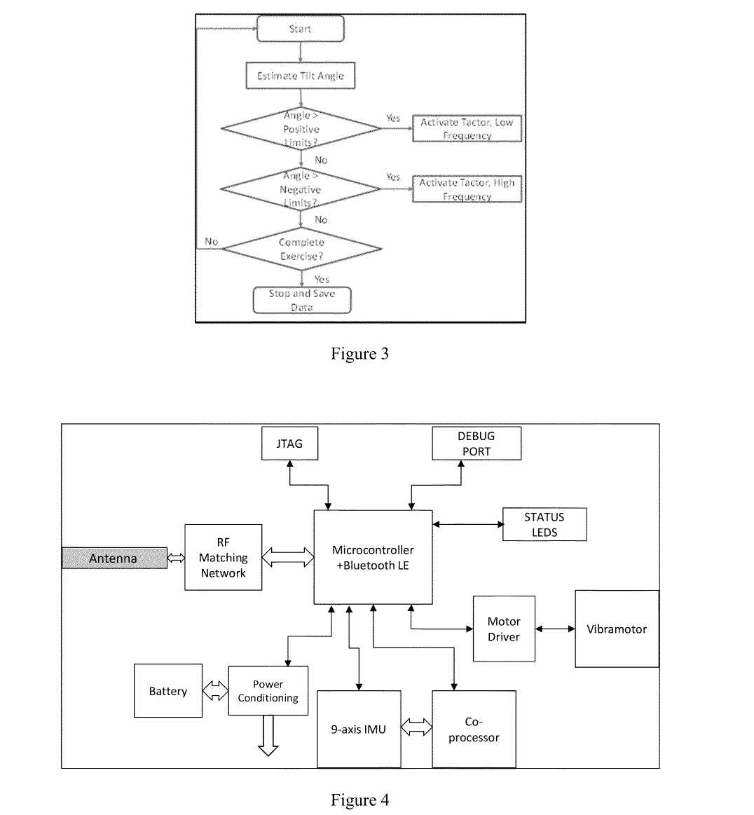

FIG. 3 depicts a flow diagram of BATS operations.

The BATS system may comprise electronics configured for sensor data acquisition, storage, and wireless telemetry. Using an industry-standard Bluetooth interface, the BATS can be provided to wirelessly interface to tablets, smartphones, and PCs. Beneficially, the BATS system may utilize commercial off the shelf or “COTS” components and standard electronic assembly techniques permitting easy migration to a production environment.

A preferred block diagram of the system is shown in FIG. 4.

Major elements of the system include an RF microcontroller System-on-Chip (SoC). Various manufactures such as T1, Nordic, and CSR produce such devices with Bluetooth (Low Energy) RF interfaces that include an 8-32bit microcontroller. The microcontroller may be configured to use a SPI serial data link to interface to the IMU to read BATS data. The IMU may be provided to have a typical maximum data rate of 1 KHz, which is easily manageable by the microcontroller.

The BATS system block diagram also illustrates a co-processor which may be an additional low-power DSP to augment the processing capabilities of the microcontroller depending on user-required algorithms.

For example, a BS300 hearing-aid processor may be used in the invention. Although such devices are intended for audio applications, they are commercially available and provide high dynamic range ADC's, multiple instructions/cycle, and consume very low power.

The microcontroller of the invention, with co-processor, may be configured to analyze sensed tilt data to activate the vibration motor to alert the user of approaching off-balance events. Also included in the BATS may be a NAND flash device configured such that the BATS system can record data and, later, allow off-line wireless download by a physician or therapy assistant. The BATS system may be configured to use the IMU data to track and store movements (and relative balance during activities such as climbing stairs or walking). Off-line analysis can desirably be used to evaluate therapy improvement and monitor patient activity.

An estimate of the BATS power consumption is summarized in Table 1 below.

TABLE 1
Preliminary power consumption power estimate

| Part | Power Consumption | Total (mW) |
|---|---|---|
| IMU (InvenSense MPU9150) | 3.9 mA @ 3.5 V | 14 |
| Wireless (TI CC2540F128) | 6.7 mA @ 2.5 V | 17 |
| Wireless (TI CC2540F128) - RF | 22.9 mA @ 2.5 V | 6 |
| | @ 10% Duty Cycle | |
| Vibration Motor | 65 mA@2 V | 13 |
| | @10% Duty Cycle | |
| Micro Processor | 5 mA @ 1.5 V | 8 |

| | |
|---|---|
| Miscellaneous Components | 5 |
| Total = | 63 mW |

For the BATS to operate effectively as a postural control device, a desired measurement accuracy of the body vertical should be within about 0.1 to 1 degree. To meet this angular measurement accuracy, gyroscopes with extremely low drift and sensitivity variations are preferred.

All gyros are designed to measure angular velocities (or angular rates). To obtain angles of rotation, the output of a gyro must be integrated with time; thus even a small output signal can lead to significant errors in measurement that increase linearly over time. One indication of the accuracy and stability of a gyro is its bias instability, which is the output rate signal when the input rate is zero, i.e., when the micro gyro is stationary.

Such stringent angular accuracy can generally only be met by using precision gyroscopes that undesirably are expensive ($1,000 to $3,000 IMU) and bulky. Moreover, extensive calibration of such gyroscopes over the full temperature range is typically required, which greatly increases the size and cost of the final device.

On the other hand, chip-scale gyroscopes (micro-gyros) are low cost ($5 to $20 IMU) and very small (millimeters size chips), but tend to have significant drift and sensitivity variations that result in large measurement errors.

Because the invention provides numerous features for addressing drift and sensitivity in chip-scale gyros, these low cost and commercially available devices are suitable for use in the device.

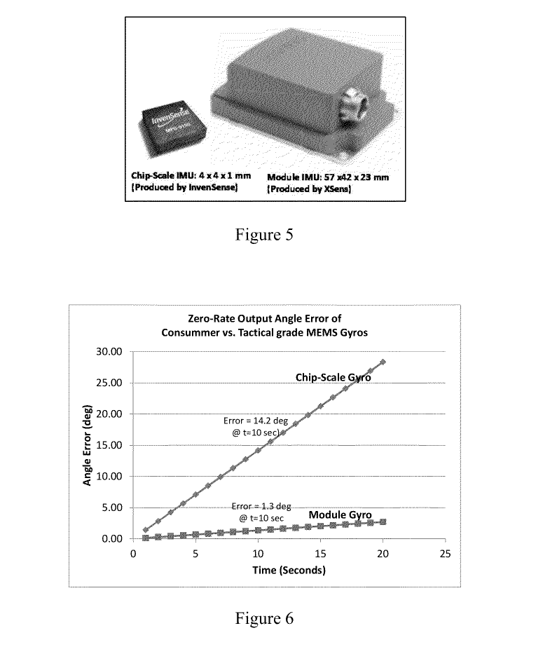

FIG. 5 shows two suitable chip-scale (InvenSense) and module (XSens) IMUs that are commercially available for use in the invention.

FIG. 6 illustrates the expected angle output over time using a precise module gyroscope (18 deg/hour bias instability) versus a chip-scale gyroscope (1,440 deg/hour bias stability).

As can be seen in FIG. 6, although the gyro is stationary, after only 10 seconds, the output error from the precise gyro is 1.3 degree, while the consumer-grade gyro is 14.2 degrees, far exceeding the 1 degree measurement accuracy needed for postural control applications.

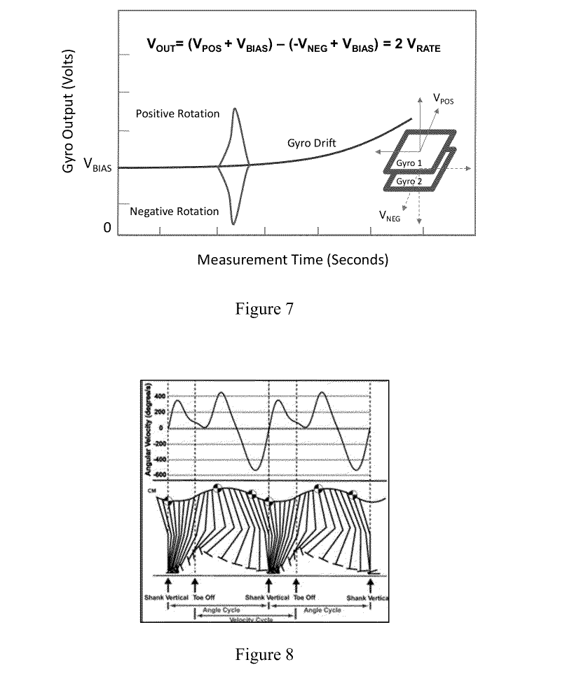

FIG. 7 illustrates how bias instability error is cancelled in the device and method of the invention. The approach herein is to take two opposing measurements of the same magnitude and cancel the bias by adding the two outputs. Although the bias will continue to change (or drift), this technique ensures that bias errors are not included in the angle measurements. Obtaining two opposing rate measurements of equal amplitude is accomplished by measuring body's natural sway motion during gait.

FIG. 8 shows how a body's angular velocities exhibit both positive and negative direction. An algorithm is executed in the device that identifies these reciprocal outputs that are closely spaced in time.

Alternatively or in addition, as mentioned above, two gyroscopes may be provided and mounted to measure in opposite directions. By providing two IMU's, positioned to measure in opposite directions, the collective drift of the system is thus actively cancelled. This configuration permits the use of low cost and small size (4×4×1 mm) of COTS chip-scale IMUs in the device.

In addition to removing the bias instability in the COTS gyroscopes of the device, a preferred embodiment of the device may include a technique for correcting the drift problem of the device by measuring the vertical angle independently from the gyroscope.

To address the drift problem, the tri-axial accelerometer (contained in IMU) is configured whereby the gravity vector is estimated.

With careful removal of lateral accelerations, and by keeping track of the initial orientations using tri-axial magnetometers, the output of the tri-axial accelerometer is put through an algorithm to compute the gravity vector. The calculated gravity vector is then used as a reference for body tilt through an Extended Kalman filter combined with gyroscope's output to improve the accuracy of the measured body tilt.

Using this technique, the output of the trunk angles as measured by accelerometers are at a relatively low bandwidth, while the gyroscope measurement are at a relatively high bandwidth.

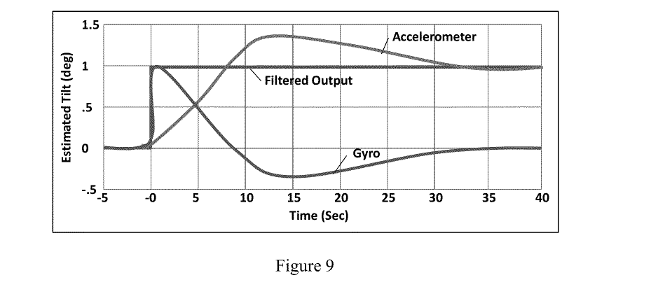

FIG. 9 illustrates the use of both an accelerometer and gyroscope to measure the body tilt over short time scales (gyroscope) and over longer time scales (accelerometer).

Maintaining postural control during quasi-static (e.g., standing, reaching) and dynamic movements (e.g., walking, turning) is central to safe mobility.

The vibratory feedback of the invention is used to alert the wearer when leaning into a potentially unstable region of the stability limits during standing tasks (e.g., reaching for objects), or when exceeding desirable body angles during the performance of dynamic movements (e.g., walking, turning, transfers).

Once mastered, the amount of augmented vibratory feedback provided during training sessions may configured to be systematically withdrawn using a fading frequency of feedback schedule. This method of feedback scheduling has been shown to be particularly effective in rehabilitation settings, resulting in both superior learning and better transfer of movement strategies to other environments.

A primary goal of balance retraining using the BATS tactile feedback capabilities is to enhance the use of anticipatory postural control strategies while minimizing the need for more complex and attentionally-demanding reactive postural control strategies.

A balance-training plan using augmented and real-time tactile feedback is preferably provided along with the wearing of the BATS to help patients relearn how to control the Center of Mass or “COM” in seated, standing, and moving environments.

The patient should be instructed on how to associate the different frequencies of augmented tactile feedback with the direction of body tilt (i.e., anterior-posterior, medial-lateral) while exploring his/her stability limits; first in a seated position, followed by standing, and moving positions once a specified criterion of mastery has been achieved.

Balance activities may include goal-directed leaning and reaching in seated and standing positions, progressing to different body transfer activities such as rising from a chair, walking and turning. As learning progresses in each balance environment, the frequency with which the tactile feedback is presented to the user may be progressively lowered.

Fading out of the frequency of the feedback facilitates the development of internal representations and a reduced dependency on the externally provided feedback. This type of balance treatment and scheduling of feedback method has been demonstrated to enhance the learning and transfer.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by any claims in any subsequent application claiming priority to this application.

For example, notwithstanding the fact that the elements of such a claim may be set forth in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings Thus, if an element can be understood in the context of this specification as including more than one meaning, then its use in a subsequent claim must be understood as being generic to all possible meanings supported by the specification and by the word itself.

The definitions of the words or elements of any claims in any subsequent application claiming priority to this application should be, therefore, defined to include not only the combination of elements which are literally set forth, but all equivalent structure, material or acts for performing substantially the same function in substantially the same way to obtain substantially the same result. In this sense, it is therefore contemplated that an equivalent substitution of two or more elements may be made for any one of the elements in such claims below or that a single element may be substituted for two or more elements in such a claim.

Although elements may be described above as acting in certain combinations and even subsequently claimed as such, it is to be expressly understood that one or more elements from a claimed combination can in some cases be excised from the combination and that such claimed combination may be directed to a subcombination or variation of a subcombination.

Insubstantial changes from any subsequently claimed subject matter as viewed by a person with ordinary skill in the art, now known or later devised, are expressly contemplated as being equivalently within the scope of such claims. Therefore, obvious substitutions now or later known to one with ordinary skill in the art are defined to be within the scope of the defined elements.

Any claims in any subsequent application claiming priority to this application are thus to be understood to include what is specifically illustrated and described above, what is conceptually equivalent, what can be obviously substituted and also what essentially incorporates the essential idea of the invention.

### REFERENCES

1. Dozza M, Chiari L, Horak F B: Audio-biofeedback improves balance in patients with bilateral vestibular loss. Arch Phys Med Rehabil 2005, 86:1401-1403.

2. Dozza M, Horak F B, Chiari L: Auditory biofeedback substitutes for loss of sensory information in maintaining stance. Exp Brain Res 2007, 178:37-48.

3. Bach-y-Rita P: Tactile sensory substitution studies. Ann NY Acad Sci 2004,1013:83-91.

4. Vuillerme N, Pinsault N, Chenu O, Demongeot J, Payan Y, Danilov Y: Sensory supplementation system based on electrotactile tongue biofeedback of head position for balance control. Neurosci Lett 2008, 431:206-210.

5. Dozza M, Wall C, Peterka R J, Chiara L, Horak F B: Effects of practicing tandem gait with and without vibrotactile biofeedback in subjects with unilateral vestibular loss. J Vestib Res 2007, 17:195-204.

6. Kentala E, Vivas J, Wall C: Reduction of postural sway by use of active vibrotactile balance prosthesis prototype in subjects with vestibular deficits Ann Otol Rhinol Laryngol 2003, 112:404-409.

7. Sienko K H, Balkwill M D, Oddsson L I, Wall C: Effects of multi-directional vibrotactile feedback on vestibular-deficient postural performance during continuous multi-directional support surface perturbations. J Vestib Res 2008, 18:273-285.

8. Wall C, Kentala E: Control of sway using vibrotactile feedback of body tilt in patients with moderate and severe postural control deficits. J Vestib Res 2005, 15:313-325.

9. Wall C, Merfeld D M, Rauch S D, Black F O: Vestibular prostheses: the engineering and biomedical issues. J Vestib Res 2003, 12:95-113.

10. Wall C, Oddsson L E, Horak F B, Wrisley D W, Dozza M: Applications of vibrotactile display of body tilt for rehabilitation. Proceedings of the 26th Annual International Conference of the IEEE Engineering in Medicine and Biology Society; San Francisco, Calif., USA 2004, 4763-4765.

11. Weinberg M S, Wall C 3rd, Robertsson J, O'Neil E, Sienko K, Fields R: Tilt determination in MEMS inertial vestibular prosthesis. J Biomech Eng 2006, 128:943-956.

12. Lee B C, Chen S, Sienko K H: A wearable device for real-time motion error detection and vibrotactile instructional cuing. IEEE Trans Neural Syst Rehabil Eng 2011, 19:374-381.

13. Sienko K H, Vichare V V, Balkwill M D, Wall C III: Assessment of vibrotactile feedback on postural stability during pseudorandom multidirectional platform motion. IEEE Trans Biomed Eng 2010, 57:944-952.

14. FreedomBalance™, Balance International Innovations [http://wwwb2i.info].

15. VertiGuard® RT, Vesticure. [http://vesticure.com].

16. Lee B C, Kim J, Chen S, Sienko H: Cell Phone Based Balance Trainer, Journal of NeuroEngineering and Rehabilitation, 2012, 9:10.

17. Wall C 3rd, Weinberg M S: Balance Prostheses for Postural Control, IEEE Engineering in Medicine and Biology Magazine, March 2003.

18. Oddsson L I E, Meyer P M: Sensor Prosthetic for Improved Balanced Control, US Patent 2005/0131317 A1.

19. Shaw C: Wireless system opens door to rapid, low cost gait analysis, News Electronics, February 2011

20. Mancini M, Horak F B; The relevance of clinical balance assessment tools to differentiate balance deficits, Eur J. Phys Rehabil Med, 2010 June; 46(2): 239-248

21. Bouchaud J:MEMS for eHealth Market Overview, 11th MEPTEC MEMS Technology Symposium, San Jose, May 2013

We claim:

1. A body worn sensor system for the purpose of balance augmentation consisting of an inertial measurement unit, a processor, a wireless radio, and a set of tactile feedback motors

2. The sensor system of claim 1 where an inertial measurement unit and a sensor fusion processor is utilized to determine the wearer's body posture, as well as angular velocities and accelerations.

3. The systems system of claim 1 where the tactile feedback motors communicate real-time body posture information to the wearer by spatial arrangement of the motors and driving waveform.

4. The sensor system of claim 1 where a wireless radio is utilized to communicate with other electronic devices.

5. The sensor system of claim 1 where a wireless radio is utilized to communicate to a separate mobile device, visually displaying real-time body posture information to the wearer.
